(19) United States
(12) Patent Application Publication
(10) Pub. No.: US 2008/0294070 A1
(43) Pub. Date: Nov. 27, 2008

(54) JUGULAR VENOUS PRESSURE GAUGE

(76) Inventor: Michael Kinori, Tel Aviv (IL)

Correspondence Address: HAIM FACTOR;Ofek Group Inc., 16192 Coastal Highway, Lewes, DE, 19958, US

(21) Appl. No.: 11/754,290

(22) Filed: May 27, 2007

(51) Int. Cl. A61B 5/103; G01C 5/00

(52) U.S. Cl. 600/587; 33/290

(57) ABSTRACT

The present invention is directed to an apparatus for metering the jugular venous pressure of a patient, and a method thereof. The apparatus comprises: a slider which comprises a scale for indicating the venous pressure of a patient; a pointer connected to the sliding member of the slider perpendicularly to the scale, for pointing on the filling level of the jugular venous pressure of the patient. The jugular venous pressure is indicated by positioning the patient at a certain angle, such as 30 to 45 degrees with reference to the floor, placing the tip at the sternal-angle of the patient while the slider is held vertically, sliding the pointer until pointing on the top of the filling level of the jugular vein of the patient.

### FIELD OF THE INVENTION

The present invention relates to the field of metering instruments for medical purposes. More particularly, the present invention relates to a gauge for measuring Jugular Venous Pressure (JVP).

### BACKGROUND OF THE INVENTION

Heart failure is a condition that can result from any structural or functional cardiac disorder that impairs the ability of the heart to fill with or pump a sufficient amount of blood throughout the body. The jugular vein provides information about right atrium and right ventricular function and venous pulsation can be detected in the jugular vein.

To diagnose heart failure, physicians regularly use two rulers to measure the height from the sternal-angle (the bony ridge adjacent to the second rib where the manubrium joins the body of the sternum) to the filling level of the jugular vein when the patient is positioned at a 45 degree angle. An elevated JVP is the classic sign of venous hypertension, e.g. right-sided heart failure. The optimal JVP measurement is to position the patient to an angle of or under 45 degrees, and measure the height of the perpendicular line forming when the bottom of the first ruler is placed on the sternal-angle with the second ruler placed at a right angle with the first, pointing at the distanced edge of the filled jugular vein. In this way, the filling level of the jugular vein is determined. In healthy people, it is 3 to 4 centimeters above the sternal-angle. Measurements of over 5 centimeters above the sternal-angle may indicate heart failure.

This type of measurement is based on the classical method for quantifying the JVP as described by Borst & Molhuysen in 1952. (Borst J, Molhuysen J. “Exact determination of the central venous pressure by a simple clinical method.” Lancet 1952 Aug. 16; 2 (7): 304-9). The method, as modified later, provides a diagnostic tool for determining heart failure by discerning the jugular filling level.

The JVP is easiest to observe if one looks along the surface of the sternocleidomastoid muscle, as it is easier to appreciate the movement relative the neck when looking from the side (as opposed to looking at the surface at a 90 degree angle). As in judging the movement of an automobile from a distance, it is easier to see the movement of an automobile when it is crossing one's path at 90 degrees (i.e., moving left to right or right to left), as opposed to coming toward one. This is a simple method to appreciate the indirectly observed pressure over the venous system.

With the present procedures of measurement, a physician must have in his hands two rulers upright each other, point one to the sternal-angle and the other to the filling level edge of the jugular vein, and at the same time measure the height of the crossing point between the two rulers. This procedure is technically complicated because the physician is supposed to see that the vertical ruler, indicating the height from the sternal-angle, is indeed perpendicular to the ground, when the patient is lying at 45 degrees to the ground. The complication of holding two rulers at the time of measurement makes the procedure awkward, and the need to keep the rulers available to the physician at all times renders the procedure even more exhausting.

It is an object of the present invention to provide a Jugular Venous Pressure (JVP) gauge, which is operated in an easy and simple way.

It is a further object of the present invention to provide a Jugular Venous Pressure (JVP) gauge which is of pocket size.

Other objects and advantages of the invention will become apparent as the description proceeds.

### SUMMARY OF THE INVENTION

The following embodiments and aspects thereof are described and illustrated in conjunction with systems, tools and methods, which are meant to be merely illustrative, not limiting in scope. In various embodiments, one or more of the above-described problems have been reduced or eliminated, while other embodiments are directed to other advantages or improvements.

In one aspect (illustrated in FIG. 1), the present invention is directed to an apparatus for metering the jugular venous pressure of a patient, the apparatus comprising:
a slider (12) comprising a scale (34) indicating the venous pressure of a patient; and
a pointer (14) connected to the sliding member (22) of the slider (12) perpendicularly to the scale, for pointing on the filling level of the jugular vein pressure of the patient.

The jugular venous pressure may be metered by positioning the patient at a certain angle (e.g., 30 degrees, 40 degrees) with reference to the floor, placing the tip at the sternal-angle of the patient while the slider is held vertically, and sliding the pointer until pointing on the top of the filling level of the jugular venous of the patient.

According to one embodiment of the invention, scale (34) indicates the distance between each point of the scale to a tip (7) of the slider (12).

According to an additional or alternative embodiment of the invention, the scale indicates normal jugular venous pressure of a patient.

According to an additional or alternative embodiment of the invention, the scale indicates abnormal jugular venous pressure of a patient.

According to one embodiment of the invention, the pointer (14) comprises a bar.

The apparatus may further comprise a spirit level (10), for indicating verticality of the scale.

The apparatus may further comprise a spirit level (10), for indicating horizontality of the pointer.

The apparatus may further comprise a dowel (21) and a corresponding depression (18), for holding the pointer perpendicularly to the scale while rendering the metering.

The apparatus may further comprise a dowel (21) and corresponding depression (19), for holding the pointer in parallel to the scale while the apparatus is idle.

In one embodiment of the invention, pointer (14) is foldable.

The apparatus may further comprise a slit (4) corresponding to the pointer, for use as housing to the pointer when folded.

The apparatus may further comprise a clip (6), for attaching the apparatus to a fabric.

The apparatus may further comprise a push button (13) for pushing the pointer 14 to protrude from the body of the slider, thereby allowing rotating the pointer 14 to be perpendicular to slider (12).

In a preferred embodiment of the invention, the slider (12) is of pocket size (i.e., small enough to be carried in a garment pocket, such as about 14 cm).

In another aspect (illustrated in FIG. 9), the present invention is directed to an apparatus for metering the jugular venous pressure (JVP) of a patient, the apparatus comprising: a ruler (12) whose scale (34) indicates the distance between each point of the scale and its projection on an horizontal plane (46), wherein the angle between ruler (12) and plane (46) is at an angle (44). According to one embodiment of the invention, the angle is substantially 30 degrees. According to another embodiment of the invention, the angle is substantially 45 degrees.

The apparatus may further comprise a spirit level (10) indicating the angle (16).

In yet another aspect, the present invention is directed to a method for metering the jugular venous pressure of a patient, the method comprising the steps of:
providing an apparatus comprising a first bar perpendicular to a second bar, the first bar comprising a scale indicating the distance between a tip thereof, the second bar slideable along the first bar;
positioning the patient at an angle, such as 30 degrees or 45 degrees, with reference to the floor; and
placing the tip of the first bar at the sternal-angle of the patient, while the first bar is held vertically sliding the second over the first bar until reaching the top of the filling level of the jugular vein of the patient;
at this position the jugular venous pressure is indicated by the value that the second bar points to on the scale.

In addition to the exemplary aspects and embodiments described above, further aspects and embodiments will become apparent by reference to the figures and by study of the following detailed description.

### BRIEF DESCRIPTION OF THE DRAWINGS

The objects and features of the present invention will become apparent from the following detailed description considered in conjunction with the accompanying drawings, in which:

FIGS. 1 and 2 illustrate a jugular venous pressure gauge, according to one embodiment of the invention. While FIG. 1 illustrates the gauge in an idle state, FIG. 2 schematically illustrates the gauge in measuring state.

FIG. 3 is an exploded view which further details the jugular venous pressure gauge of FIGS. 1 and 2.

FIG. 4 is a sectional view of cross-section A-A of FIG. 6.

FIG. 5 is a sectional view of cross-section B-B of FIG. 6.

FIG. 6 is a top view of the jugular venous pressure gauge of FIGS. 1 and 2. It specifies two cross-sections, A-A and B-B.

FIG. 7 illustrates a jugular venous pressure gauge, according to another embodiment of the invention.

FIG. 8 pictorially illustrates a measurement carried out by the jugular venous pressure gauge of FIG. 1.

FIG. 9 schematically illustrates a jugular venous pressure gauge, according to another embodiment of the invention.

FIG. 10 pictorially illustrates a measurement carried out by a jugular venous pressure gauge of FIG. 9.

It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein. Reference numerals may be repeated among the figures to indicate corresponding or analogous elements.

### DETAILED DESCRIPTION OF PREFERRED EMBODIMENTS

In the following detailed description, numerous specific details are set forth in order to provide a thorough understanding of the disclosure. However, it will be understood by those skilled in the art that the present invention may be practiced without these specific details. In other instances, well-known methods, procedures, components and circuits have not been described in detail, so as not to obscure the present disclosure.

### The Structure of One Embodiment

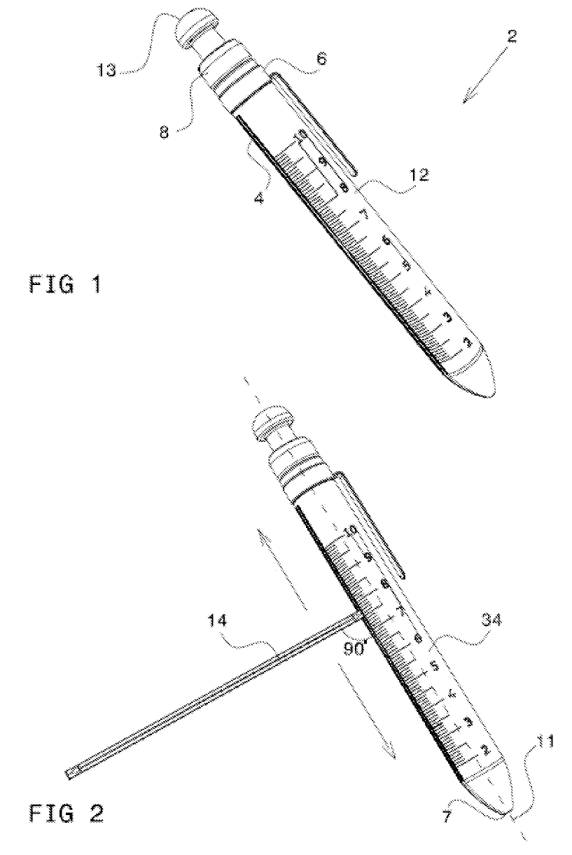

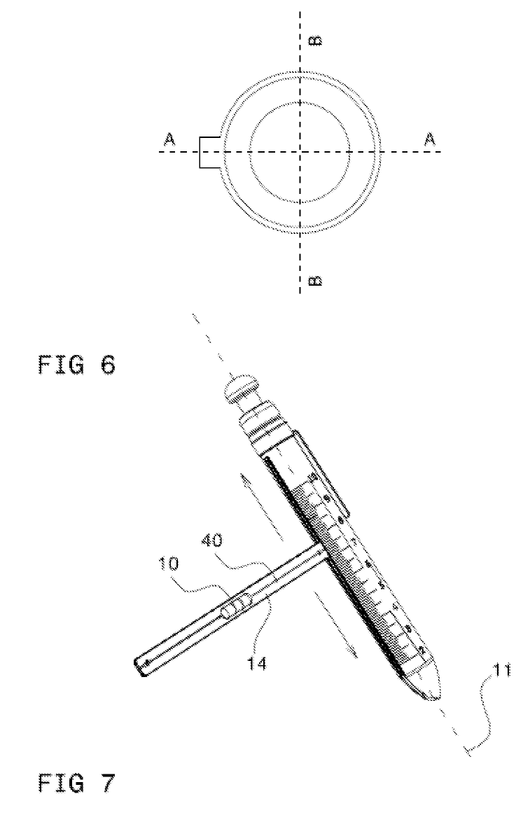

In the embodiments illustrated in FIGS. 1 to 8, the gauge is embodied as a pen, having two states: an idle state, and a measuring state.

FIG. 1 schematically illustrates a jugular venous pressure gauge in its idle state, in accordance with a preferred embodiment of the present invention.

FIG. 2 schematically illustrates the jugular venous pressure gauge of FIG. 1 in its measuring state.

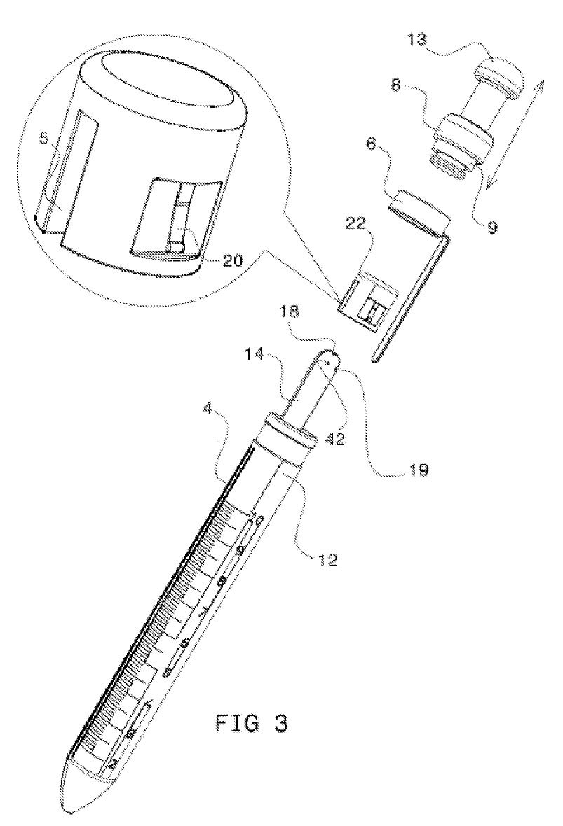

FIG. 3 is an exploded view of the jugular venous pressure gauge of FIG. 1.

In these figures, gauge 2 is designed as a pocket-size pen.

Scale 34 denotes the distance between tip 7 of gauge 2 and pointer 14. The JVP is commonly metered by centimeters. As such, in FIGS. 1 to 7 the scale 34 is in centimeters, i.e., according to the metric system, but any other scale may be used.

The basic structure of the embodiments illustrated in FIGS. 1 to 9 is two parts: a body 12 and a pointer 14 which slides along body 12. In order to measure the JVP of a patient by gauge 2, pointer 14 should be placed perpendicularly to body 12. In this situation, i.e., when axis 11 of body 12 and pointer 14 are perpendicular, pointer 14 slides along axis 11 of body 12. The arrows in FIG. 2 illustrate the direction of sliding.

Element 22 is a cylinder whose external diameter is slightly smaller than the diameter of body 12 in order to be slideable along body 12. Since cylinder 22 resembles a piston, it is also referred to herein as piston. Pointer 14 is connected to piston 22 by an axle 42. Thus, pointer 14 may be rotated around axle 42.

Element 20 of cylinder 22 is a spring, which tightens cylinder 22 to the interior of body 12.

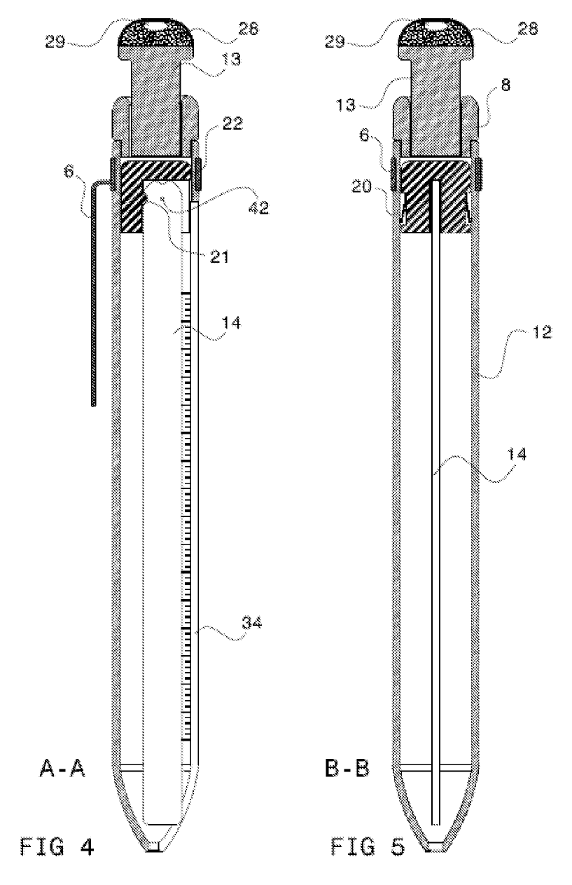

Element 22 also comprises a dowel 21 (seen in FIG. 4), which corresponds to depressions 18 and 19 of pointer 14, thereby providing two stop points: a stop point at which the angle between axis 11 of body 12 and pointer 14 is 0 degrees, and a stop point at which the angle between axis 11 of body 12 and pointer 14 is 90 degrees. In the measuring state, the angle between axis 11 and pointer 14 is 90 degrees, and in the idle state the angle is 0 degrees.

Body 12 comprises a slit 4 which is used as housing for pointer 14 in the idle state of the gauge.

Element 8 is a threaded cylinder. The threads 9 of cylinder 8 are used for securing cylinder 8 to body 12.

Element 13 is a push button whose external diameter is slightly smaller than the inner diameter of cylinder 8, thereby being slideable along cylinder 8. When the gauge is in the idle state (i.e., folded), pushing push button 13 inside the body results in exposing the tip of pointer 14, thereby allowing to position pointer 14 to be perpendicular to body 12. Of course, pointer 14 may be designed to be somewhat longer, thereby outstanding from body 12.

In the embodiment of FIG. 1, a spirit level 10 is attached to one end of push button 13. The spirit level 10 helps keep body 12 vertical while carrying out a measurement. According to one embodiment of the invention, spirit level 10 is a tube. According to another embodiment of the invention, spirit level 14 is a sphere. The difference between a tube and a sphere is that while the tube indicates a tilt in one plane, a sphere indicates a tilt in the space.

While in the embodiment illustrated in FIGS. 1 and 2 the spirit level is attached to the push button 13, in the embodiment illustrated in FIG. 7 the spirit level is attached to pointer 14.

FIG. 7 schematically illustrates a jugular venous pressure gauge, in accordance with another preferred embodiment of the present invention.

In this embodiment of the invention, the gauge comprises spirit-level 10 which is placed on the pointer, in contrast to the embodiment of FIGS. 1 and 2 in which the spirit level is placed on body 12 of the gauge.

### Measuring the JVP

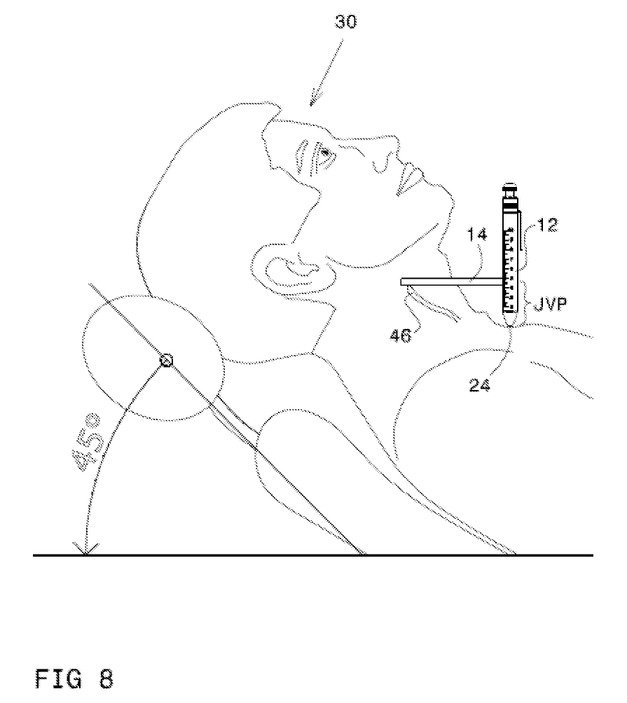

FIG. 8 illustrates a measurement carried out by the gauges illustrated in FIGS. 1 to 7.

Firstly, the patient is placed at a tilt of 45 degrees with the floor. It should be noted that some physicians prefer a tilt of 30 degrees.

While rendering the measurement, pointer 14 must be perpendicular to the axis of the body. Thus, a physician must rotate pointer 14 until body 12 and pointer 14 are perpendicular.

While holding the gauge vertically (this can be indicated by the spirit level 10), the physician places the tip of the gauge's body 12 on sternal-angle 24 of patient 30.

While holding the gauge on the sternal-angle 24, the physician slides pointer 14 along body 12 until the pointer 14 is at the filling level edge of the jugular vein (marked by numeral 46).

At this time, the value that pointer 14 points at is the JVP.

### The Structure of Another Embodiment

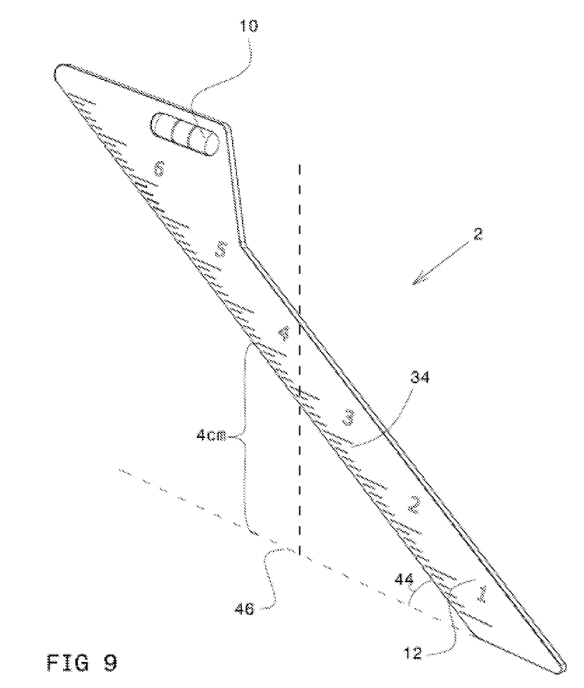

FIG. 9 schematically illustrates a jugular venous pressure gauge, according to another embodiment of the invention.

According to this embodiment, the gauge 2 is based on a ruler 12 whose scale 34 indicates the distance between a point thereon and a plane 46 in a situation wherein the ruler 12 is tilted at a degrees with the plane. As mentioned above, preferably α is 45 degrees, but some physicians prefer an angle of 30 degrees.

A spirit level 10 may be added to ruler 12 in order to indicate the desired angle α.

Since the scale indicates the distance between a point thereon and a plane that tilts in α degrees with the ruler, the physical distance of 1 cm of the scale is 1/sin α.

As mentioned above, some physicians prefer to measure the JVP while the patient is tilted at 30 degrees with the floor. Accordingly, the gauge may be designed to correspond to 30 degrees rather than 45 degrees, or to correspond with any desirable degree.

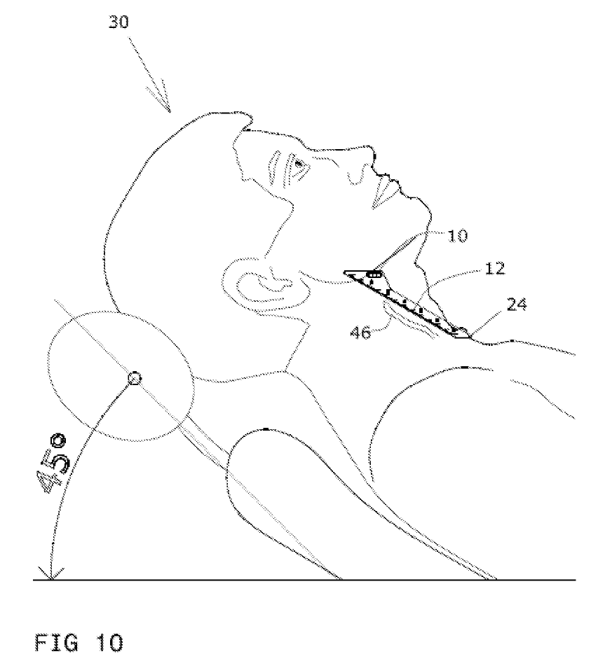

FIG. 10 pictorially illustrates a measurement carried out by a jugular venous pressure gauge of FIG. 9.

The patient is placed at a tilt of 45 degrees with the floor. As mentioned, some physicians prefer a tilt of 30 degrees.

While holding the gauge at the desired angle (45 or 30 degrees), the physician places the tip of the gauge's body 12 on sternal-angle 24 of patient 30, and rotates the gauge along the tip until spirit level 10 indicates that the patient is at the desired angle.

At this situation the filling level edge of the corresponding point of the jugular vein (marked by numeral 46) on scale 34 indicates the JVP.

### The Scale

In the embodiments illustrated herein, the scale indicates the JVP. Additionally or alternatively, a scale may indicate an acceptable or unacceptable result. For example, the range of acceptable JVP values may be in a different color than the scale (for example, acceptable value in green, possibility of heart failure in red, etc.)

In the description and figures herein, the following numerals are mentioned:
Numeral 2 denotes a jugular venous pressure gauge, according to embodiments of the present invention;
Numeral 4 denotes a slit in the body 12 of gauge 2, which serves as housing for pointer 14;
Numeral 5 denotes a slit in cylinder 22;
Numeral 6 denotes a clip;
Numeral 7 denotes a tip of gauge 2;
Numeral 8 denotes a threaded cylinder;
Numeral 9 denotes the threads of cylinder 8;
Numeral 10 denotes a spirit level, for indication that the axis 11 of gauge 2 is vertical;
Numeral 11 denotes the axis of pen-gauge 2;
Numeral 12 denotes the body of the pen-gauge 2;
Numeral 13 denotes a push button whose external diameter is slightly smaller than the inner diameter of cylinder 8, thereby being slideable along cylinder 8;
Numeral 14 denotes a bar used as a pointer;
Numeral 18 denotes a depression which corresponds to dowel 21;
Numeral 19 denotes a depression which corresponds to dowel 21;
Numeral 20 denotes an element which operates as a spring that presses element 20 to the walls of the interior of body 12, and also presses element 20 towards pointer 14;
Numeral 21 denotes a dowel which corresponds to depressions 18 (secures pointer 14 in the measure state) and 19 (secures pointer 14 in the idle state);
Numeral 22 denotes a cylinder (referred to herein as piston) whose diameter is slightly smaller than the interior diameter of body 12;
Numeral 24 denotes the sternal-angle of a patient;
Numeral 28 denotes the fluid of the spirit level 10;
Numeral 29 denotes the bubble of the spirit level 10;
Numeral 30 denotes a patient;
Numeral 34 denotes a scale which indicates the distance between the tip of gauge 2 (marked as 7) and pointer 14;
Numeral 42 denotes an axle which connects pointer 14 to piston 22;
Numeral 44 denotes the angle between ruler 12 and the projection thereof on a horizontal plane 46 (illustrated in FIG. 10); and
Numeral 46 denotes a horizontal plane (illustrated in FIG. 10).

While certain features of the invention have been illustrated and described herein, the invention can be embodied in other forms, ways, modifications, substitutions, changes, equivalents, and so forth. The embodiments described herein should be considered as illustrative and not restrictive.

What is claimed is:

1. An apparatus for metering the jugular venous pressure of a patient, the apparatus comprising:
a slider comprising a scale indicating the jugular venous pressure of a patient; and
a pointer, connected to a sliding member of said slider perpendicularly to said scale, for pointing on the filling level of the jugular vein of said patient.

2. An apparatus according to claim 1, wherein said scale indicates the distance between each point of said scale to a tip of said slider.

3. An apparatus according to claim 1, wherein said scale indicates normal jugular venous pressure of a patient.

4. An apparatus according to claim 1, wherein said scale indicates abnormal jugular venous pressure of a patient.

5. An apparatus according to claim 1, wherein said pointer comprises a bar.

6. An apparatus according to claim 1, further comprising a spirit level, for indicating verticality of said scale.

7. An apparatus according to claim 1, further comprising a spirit level, for indicating horizontality of said pointer.

8. An apparatus according to claim 1, further comprising a dowel and a corresponding depression, for holding said pointer perpendicularly to said scale while rendering said metering.

9. An apparatus according to claim 1, further comprising a dowel and corresponding depression, for holding said pointer parallel to said scale while said apparatus is idle.

10. An apparatus according to claim 1, wherein said pointer is foldable.

11. An apparatus according to claim 10, further comprising a slit corresponding to said pointer, said slit being used as housing for said pointer when folded.

12. An apparatus according to claim 1, further comprising a clip, for attaching said apparatus to a fabric.

13. An apparatus according to claim 1, further comprising a push button for pushing said pointer to protrude from the body of said slider.

14. An apparatus according to claim 1, wherein the length of said first bar is of pocket size.

15. A method for metering the jugular venous pressure of a patient, the method comprising the steps of:
providing an apparatus comprising a first bar perpendicular to a second bar, said first bar comprising a scale indicating the distance between a tip thereof, said second bar slideable along said first bar;
positioning said patient at an angle with reference to the floor; and
placing said tip at the sternal-angle of said patient while said first bar is held vertically, sliding said pointer over said first bar until reaching the top of the filling level of the jugular vein of said patient.

16. A method according to claim 15, wherein said angle is substantially 30 degrees.

17. A method according to claim 15, wherein said angle is substantially 45 degrees.

18. A method according to claim 15, wherein said apparatus further comprise a spirit level for indicating the verticality of said apparatus.

19. An apparatus for metering the jugular venous pressure of a patient, the apparatus comprising:
a ruler the scale of which is adapted to indicate the distance between each point of said scale and its projection thereof on a plane while said ruler is in an angle with said plane.

20. An apparatus according to claim 19, further comprising a spirit level indicating said angle.
